(19) United States
(12) Patent Application Publication
(10) Pub. No.: US 2018/0221679 A1
(43) Pub. Date: Aug. 9, 2018

(54) CATHETER ASSEMBLY AND COMPONENTS THEREOF

(71) Applicant: Dabney Patents, L.L.C., Georgetown, TX (US)

(72) Inventor: Paul DABNEY, Georgetown, TX (US)

(73) Assignee: Dabney Patents, L.L.C., Georgetown, TX (US)

(21) Appl. No.: 15/943,953

(22) Filed: Apr. 3, 2018

Related U.S. Application Data
(63) Continuation-in-part of application No. 14/583,580, filed on Dec. 26, 2014, now Pat. No. 9,931,189.
Application No. 14/583,580 is a continuation-in-part of application No. 14/497,269, filed on Sep. 25, 2014, now Pat. No. 9,579,177.
Application No. 14/583,580 is a continuation of application No. 14/536,633, filed on Nov. 9, 2014, now Pat. No. 9,504,848.
(60) Provisional application No. 62/026,498, filed on Jul. 18, 2014.

(51) Int. Cl. A61N 5/06; A01N 59/00; A61C 19/06; A01N 25/34

(52) U.S. Cl. CPC A61N 5/062; A61N 5/0624; A01N 59/00; A61C 19/063; A61N 2005/0647; A01N 25/34; A61N 2005/063; A61N 2005/0636; A61N 2005/0645; A61N 5/0601

(57) ABSTRACT

According to an exemplary embodiment, a catheter assembly may be provided. The catheter assembly may include: an elongated, tubular catheter body having a proximal end, a distal end, and a centrally-disposed lumen extending therebetween; a covering attached to the distal end of the catheter body, the covering defining a generally spherical shaped tip; at least one ancillary lumen disposed within the catheter body and extending between the proximal end and the distal end thereof; at least one light-emitting element configured to emit light of a predetermined wavelength through the ancillary lumen; and at least one light source that provides light of a predetermined wavelength to the at least one light-emitting element. The catheter assembly may emit certain wavelengths of light that, when combined with a target therapeutic solution, initiates a synergistic reaction.

### RELATED APPLICATIONS

This application claims the benefit of the filing date of U.S. Patent Application No. 62/026,498, filed Jul. 18, 2014, which is incorporated herein by reference in its entirety; U.S. patent application Ser. No. 14/497,269, filed Sep. 25, 2014, which is incorporated herein by reference in its entirety; U.S. patent application Ser. No. 14/536,633, filed Nov. 9, 2014, which is incorporated herein by reference in its entirety; and U.S. patent application Ser. No. 14/583,580, filed Dec. 26, 2014, which is incorporated herein by reference in its entirety.

### BACKGROUND

The present invention relates generally to therapeutic drug administration, and in particular to a catheter assembly for enhanced site-specific treatment.

Medicaments are commonly introduced into the body by various anatomical routes. Delivery methods are generally classified by the location at which the medicament is introduced to a patient and the target action thereof. Routes of drug administration commonly include, for example, enteral (e.g., oral ingestion), parenteral (e.g., intravenous, subcutaneous, and intramuscular injection), and topical course. Action may be intended for wide-spread effect or targeted to specific organs and diseases.

Traditionally, the administration of therapeutic agents to treat various medical conditions occurred via systemic delivery. Systemic delivery refers to treatment that affects the body as a whole, or that acts specifically on systems that involve the entire body. An active agent administered systemically enters the blood or lymphatic supply at a discrete location, and subsequently travels throughout the patient's body to reach and affect the diseased area. While convenient and efficient for treating multiple sites simultaneously, systemic delivery exposes non-target areas, e.g., healthy tissue, to actions of the drug. Additionally, medicines administered systemically may be more vulnerable to metabolic degradation, necessitating higher initial doses than are required for a therapeutic amount.

Local drug delivery, on the other hand, can reduce the risks associated with systemic toxicities. In contrast to conventional systemic release, local drug delivery involves the administration of a therapeutic agent directly to the target site, i.e., the tissue or organ for which treatment is sought. Local drug delivery limits action of the drug to selective regions of the body, enabling greater control of concentration levels and duration of therapeutic exposure.

Many techniques exist for the introduction of therapeutic compositions to targeted patient sites. Catheter technology, in particular, has become increasingly common in both systemic and local drug delivery systems. A catheter is a tubular medical device that can be inserted into a body cavity, duct, or vessel as a direct conduit for administering drugs and other therapeutic/diagnostic agents to infected bodily tissue. Significantly, a catheter can provide acute location deployment for medical intervention.

Light of certain wavelengths has been demonstrated to improve or “super-charge” the effects of certain pharmaceuticals or target chemicals, such as antimicrobial and antineoplastic agents, creating a synergistic effect to destroy or inhibit microbial or neoplastic growth. The development of a novel catheter assembly that induces a synergistic reaction using a certain wavelength of light to amplify the effects of certain chemicals would be an advance in the art. Increasing the effectiveness of a drug by the addition of a certain wavelength of light is a means of modifying its biological properties. It would be particularly advantageous to place these elements internally in human or veterinary patients to establish the synergistic reaction within a targeted body site. As yet, there is no catheter-associated treatment that includes a synergistic reaction between light of a certain wavelength and a chemical or chemicals.

A need thus exists for a catheter assembly that can supply a radiation source and a target chemical to a specific body region. The catheter will deliver these components while also combining flexibility and maneuverability that permits safe and easy introduction into the heart, kidney, liver, or any other targeted location where a desired synergistic chemical reaction between a certain wavelength of radiation and a chemical is needed. By placing the catheter assembly in a desired location, a localized treatment can be obtained. By utilizing other locations, a systemic treatment can be obtained.

### SUMMARY

Exemplary embodiments described herein may relate generally to therapeutic drug administration, and in particular to a system, method, and apparatus for delivering therapeutic treatment to the bodily tissue of a human or veterinary patient.

According to an exemplary embodiment, a catheter assembly may be provided. The catheter assembly may include: an elongated, tubular catheter body having a proximal end, a distal end, and a centrally-disposed lumen extending therebetween; a covering attached to the distal end of the catheter body, the covering defining a generally spherical shaped tip; at least one ancillary lumen disposed within the catheter body and extending between the proximal end and the distal end thereof; at least one light-emitting element configured to emit light of a predetermined wavelength through the ancillary lumen; and at least one light source that provides light of a predetermined wavelength to the at least one light-emitting element. The catheter assembly may emit certain wavelengths of light that, when combined with a target therapeutic solution, initiates a synergistic effect. The solution may be “super-charged” by the wavelengths of light, causing the solution to eliminate or reduce microorganisms at a higher percentage than the solution acting alone.

According to another exemplary embodiment, a method of localized therapeutic treatment may be provided. The method may include: introducing a catheter assembly into a biological conduit (vessel) of a patient; guiding the catheter assembly to a specific anatomical region for treatment therein; administering a therapeutic solution through a lumen of the catheter; selecting a wavelength of light that reacts with the administered therapeutic solution to cause a synergistic reaction; providing the selected wavelength of light through a lumen of the catheter; and irradiating the therapeutic solution with the light.

### BRIEF DESCRIPTION OF THE FIGURES

Advantages of embodiments of the present invention will be apparent from the following detailed description of the exemplary embodiments. The following detailed description should be considered in conjunction with the accompanying figure in which:

FIG. 1 is a perspective view of a catheter assembly according to an exemplary embodiment of the present invention; and

FIG. 2 is a partially sectional view of an exemplary embodiment of the catheter assembly at the distal end thereof.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Exemplary embodiments described herein may relate generally to therapeutic drug administration, and in particular to a system, method, and apparatus for providing localized treatment to the bodily tissue of a human or veterinary patient. As discussed in U.S. patent application Ser. No. 14/583,580, the contents of which are hereby incorporated in their entirety, light of certain wavelengths may improve or “super-charge” the effects of therapeutic agents, creating a synergistic effect to destroy or inhibit microbial growth. For example, an antimicrobial solution or solutions may be activated or combined with certain wavelengths of light to eliminate or reduce microbes at a higher percentage than by the solution alone. In one exemplary embodiment, a synergistic effect between certain wavelengths of light and antimicrobial solution can result in the elimination or reduction of diseases and other undesired health conditions caused by microorganisms. Exemplary embodiments described herein can provide for more effective therapeutic results when used in accordance with the methods, systems, and apparatuses described below.

Microbes exist that cause harm or disease in living tissues. By adding light of certain wavelengths to a device that holds certain therapeutic agents in close proximity to tissues, a synergistic effect can be created to destroy or inhibit microbial growth. For intracorporeal therapy, this device may be a catheter assembly designed to cover infected tissues, tumors, and other biological materials within the body cavity. The catheter assembly may increase the efficacy of therapeutic agents in contact with tissue by initiating a synergistic reaction. The catheter assembly may emit certain wavelengths of light that, when combined with certain therapeutic agents, cause a synergistic antimicrobial effect. The light may be produced, for example, from a light emitting diode (LED) or laser. An external light source may be connected to a light-emitting element, such as a fiber optic cable, with a fiber optic connection cable that may also include a fiber optic connection interface or plug.

As used herein, the term “therapeutic agent” refers to any biologically-active chemical or natural substance useful for treating a medical or veterinary disorder, preventing a medial or veterinary disorder, or regulating the physiology of a human or veterinary patient. Examples, without limitation, may include an antibiotic, antimicrobial agent, antifungal agent, antiviral agent, antiparasitic agent, anti-neoplastic agent, antioxidant, and the like, and any combination thereof. The therapeutic agent may be combined with any suitable solvent to form a therapeutic solution.

Embodiments of the present invention may generally provide medical and veterinary applications of light to a therapeutic solution containing antimicrobial or antineoplastic agents. Applications may increase the efficacy of therapeutic agents in contact with tissues, thereby eliminating or greatly reducing microbial and/or neoplastic growth of disease-causing microorganisms.

In accordance with the present invention, a biocompatible medical device may deliver a therapeutic agent or combination of agents, to a targeted region of tissue while simultaneously exposing the tissue and/or agents(s) to light of a predetermined wavelength. The therapeutic agent may be activated or combined with the light to eliminate or reduce microbes or neoplasms with a greater efficiency than the agent acting alone. The biocompatible medical device may form a catheter assembly that can advance through a patient's vasculature to a target site of action. In some exemplary embodiments, the catheter assembly may infuse therapeutic solution into a patient while simultaneously exposing said solution to light of a predetermined wavelength. The localized administration may maximize a pharmacological response at the target site of action while preventing undesirable interaction at other sites within the body.

In other exemplary embodiments, the therapeutic agent may be introduced into the cardiovascular system of a patient separately from the catheter assembly via a systemic route of administration. For example, the agent may be delivered via parenteral administration (including subcutaneous, intramuscular, and intravenous routes) or enteral administration (including oral, gastric, and rectal routes). After introducing the therapeutic agent to the patient, a catheter assembly may be guided to a specific region of treatment to emit light of certain wavelength(s) that causes a synergistic reaction with the absorbed agent. “Super charging” the agent with certain wavelengths of lights may cause the agent to eliminate or reduce microbes at a higher percentage than the agent acting alone.

Embodiments may utilize light in the violet/blue spectral region, or any wavelength of light that causes a synergistic reaction with an administered therapeutic agent from exposure lasting a few seconds to minutes. Embodiments of therapeutic agents may be used in tablet, pill, capsule, gel, liquid, spray, mist, cream, or paste form. Embodiments may be used at varying temperatures to modulate their efficacy.

In some exemplary embodiments, the therapeutic solution may include a light activated pigment that may fluoresce when exposed to the wavelength of light used in the treatment. This pigment may indicate to the physician that the synergistic reaction is occurring.

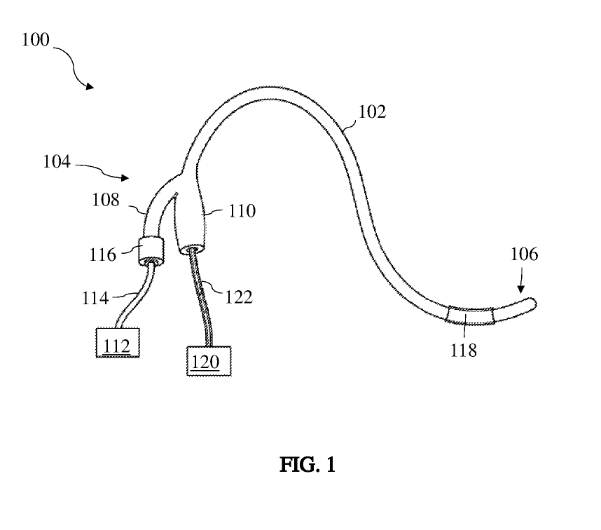

Referring now to the figures, FIG. 1 may illustrate a perspective view of a catheter assembly 100 according to an exemplary embodiment of the present invention. The catheter assembly 100 may include a catheter body formed of an elongated tubular shaft 102 having a proximal end 104, a distal end 106, and a centrally-disposed lumen extending therebetween. The catheter body may be composed of a medical grade plastic, such as polyethylene, polyvinylchloride, polyurethane, and the like, or any other conventional biocompatible material as would be understood by a person having ordinary skill in the art.

The proximal end 104 of the shaft 102 may diverge into a number of different entry ports 108, 110, each port in communication with an ancillary lumen defined within the shaft 102. The entry ports 108, 110, in conjunction with supplemental attachments, may enable a variety of medical and/or diagnostic procedures, including but not limited to, multiple site drainage or administration of therapeutic substances. While two entry ports 108, 110 are shown in FIG. 1, it should be appreciated that any number of entry ports and/or ancillary lumen can be utilized as best determined by actual operating requirements.

In one exemplary embodiment, for example, port 108 may be adapted to receive a light-emitting element such as a fiber optic cable, OLED fibers, LED strand, or other optical waveguide capable of transmitting light along a length of the catheter assembly 100. An external light source 112 may connect to the light-emitting element via a connection cable 114 and a connection interface 116. An embodiment of the interface may include a fiber optic connection cable fixed to the fiber optic cable. Another embodiment of the interface may include a socket that mates with a plug on the connection cable so that the light source can be attached and removed after use.

The light-emitting element may transmit light of a predetermined wavelength or frequency from the light source 112 to a target area within a patient's body. Light may exit the catheter shaft 102 through outlet 118. The light source 112 may comprise a single source of monochromatic light, such as a laser, or a source capable of two or more wavelengths, such as a light-emitting diode (LED). The light-emitting element of the catheter may be the same diameter as the rest of the catheter or it may be larger or smaller in diameter. The light-emitting element of the catheter may be placed at the tip of the catheter or it may exist along the entire length thereof.

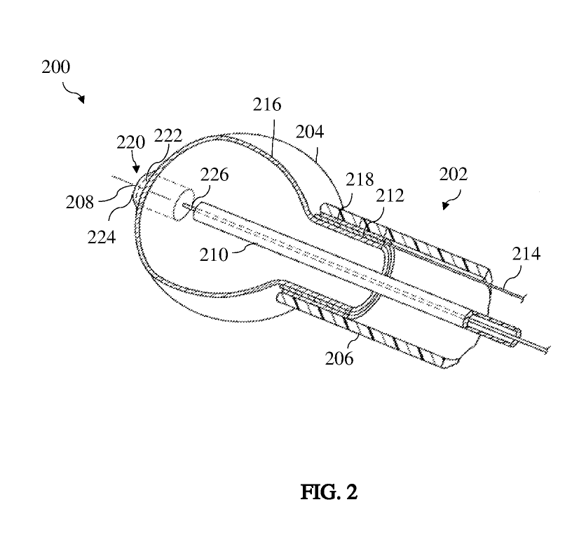

Entry port 110 may be adapted for administering therapeutic agents and/or solutions to the targeted patient site. The entry port 110 may communicate with a drug delivery lumen extending within the catheter shaft 102 from the entry port 110 to an outlet 118. Outlet 118 may be embodied by various types of openings, including, but not limited to, holes, slits, gaps, porous membranes, and osmotic filters. In one exemplary embodiment, for example, the outlet may form a mesh screen that enables the passage of therapeutic solution at a predictable flow rate thereacross. In another exemplary embodiment, the outlet 118 may take the form of an inflatable balloon situated circumferentially around the catheter shaft 102. The balloon may be inflated by way of the drug delivery lumen, whereby the inflation pressure results in infusion of the therapeutic solution through apertures in the inflatable material. As shown in FIG. 1, the outlet 118 may be positioned proximate the distal end 106 of the catheter shaft 102. In other exemplary embodiments, and as shown in FIG. 2, the outlet may be located at the tip of the catheter shaft.

A reservoir 120 may store the therapeutic agent or solution prior to use with the catheter assembly 100. The reservoir 120 may connect to the entry port 110 through an input tube 122. Input tube 122 may extend the length of the catheter assembly 100 to administer the therapeutic agent through the outlet 118, or may alternatively connect to a separate drug delivery lumen provided in the catheter body.

The catheter assembly 100 may incorporate a temperature-controlling mechanism to maintain a desired temperature of the therapeutic solution. The temperature-controlling mechanism may contain a heating element, a cooling element, or preferably both. Exemplary heating elements may include, for example, resistive wires, thin films, heating coils, radiant heat sources, or any other suitable heating means as would be understood by a person having ordinary skill in the art. Exemplary cooling elements may include a Peltier element, thermoelectric cooling chip, coolant fluid source or any other suitable cooling means as would be understood by a person having ordinary skill in the art.

In some exemplary embodiments, the temperature-controlling mechanism may be integrated into the catheter assembly 100, such as running adjacent to the light-emitting element in a separate, preformed lumen. Some embodiments of integrated temperature elements may extend only a portion of the device, such as within an entry port or a partial length of the catheter shaft 102. In other exemplary embodiments, for example, a heating filament may be inserted in a preformed catheter lumen or incorporated into a wall of the catheter shaft 102. Temperature-controlling mechanism may draw power from the same source as the external light source 112. Power may be supplied to the temperature-controlling mechanism in the device through the fiber optic connection cable 114 or through a power connection cable that runs alongside the connection cable. Power supply may be any apparatus known to those of skill in the art, including batteries, DC power unit, generators, solar power, AC power supplies or combinations thereof.

Alternate exemplary embodiments may maintain the temperature-controlling mechanism separately from the catheter assembly 100. For instance, separate heating elements may warm the therapeutic solution to an optimal temperature before the solution is added to the catheter assembly 100, such as with a heating tray or oven. In other exemplary embodiments, the temperature-controlling mechanism may be incorporated into the reservoir 120 and utilized while the therapeutic solution is stored therein.

As illustrated in FIG. 1, the catheter assembly 100 may bifurcate into single lumen lines at entry ports 108, 110. A standard medical fitting may attach to the opening of each entry port 108, 110 for connecting various medical equipment thereto. Entry ports 108, 110 may be compatible with electrical, chemical, electrochemical, temperature and/or pressure devices which enable the observation and analysis of tissue, and any other suitable medical attachment as would be understood by a person having ordinary skill in the art.

In some exemplary embodiments, an entry port may attach to a source of negative pressure to form an aspiration port for transporting fluid and obstructive material withdrawn from a body vessel. The source of negative pressure may be, for instance, a vacuum, syringe, mechanical pump, or any other source of negative pressure as would be understood by a person having ordinary skill in the art. In some exemplary embodiments, an entry port may be used as a conduit for circuitry and other components relating to one or more physiologic sensors. The sensors may be distally mounted on the catheter shaft 102 for detecting a property indicative of pressure, pH, temperature, etc. It should be understood that these examples are merely illustrative, and that any other suitable medical instrument or attachment could be used or otherwise implemented in conjunction with the catheter assembly.

FIG. 2 may illustrate an exemplary embodiment of a catheter assembly 200 at the distal end 202 thereof. A covering 204 may attach to the distal end 202 of the catheter shaft 206 to define a generally spherical-shaped tip. The covering 204 may be a transparent, protective lens having at least one aperture 208 therein. The at least one aperture 208 may be centrally located in the covering 204, and capable of dispensing therapeutic solution from a drug delivery lumen 210. It may be contemplated that any number of suitable drug delivery lumen and corresponding apertures exist in the catheter design according to the requirements of a particular application.

The catheter assembly 200 may also include at least one ancillary lumen 212 extending from the proximal end (not shown in FIG. 2) to the distal end 202 of the catheter shaft 206. The ancillary lumen 212 may be configured to receive at least one flexible, elongated light-emitting element 214. The light-emitting element 214 may be, for example, a fiber optic cable having a number of light terminations along the length thereof. The fiber optic cable may be opaque or may be at least partially translucent to emit light along its length.

In some exemplary embodiments, the light terminations may be positioned at, or proximate, the distal end of the catheter shaft 206, near the aperture 208, so that the light shines onto the therapeutic solution while the solution is being dispensed into the body. In other exemplary embodiments, the therapeutic solution may be light-enhanced prior to use with the catheter. The light terminations of the fiber optic cable may be adjustable, so that each can be added, removed, or relocated as seen fit. The light provided to the light-emitting element may include at least one frequency of light. In some exemplary embodiments, the light may include at least two frequencies of light that are simultaneously applied to a therapeutic agent or solution, or a first frequency of light may be applied and then a second frequency of light may be applied in series. Each of the frequencies of light may cause a synergistic reaction with a target therapeutic agent.

A reflective surface 216 may be positioned within the covering 204 to aid in directing light from the light-emitting element 214 to the target site. The configuration of the reflective surface 214 may be customizable depending on the treatment. For example, the reflective surface 214 may disperse the light over a wide area or may concentrate the light to a guided point.

The catheter assembly 200 may further include at least one heat transfer lumen 218 for accommodating the temperature-controlling mechanism. The temperature-controlling mechanism may be integrated into the catheter assembly 200, such as running adjacent to the light-emitting element 214 in a separate, preformed lumen 218.

In some exemplary embodiments, the catheter assembly may include a camera 220 disposed at the distal end 202 of the catheter shaft 206 for providing visual feedback to the operating physician. The camera 220 may contain an adjustable lens 222, which may be remotely operated, and/or a covering 224. The camera 220 may attach to a transmission cable 226 extending within a lumen defined by the drug delivery lumen 210, and may be positioned concentric to the aperture 208 in the covering 204. The camera 220 may provide real-time video feedback or still images to an external monitor so that the physician may directly visualize the operation as performed.

A method of providing therapeutic treatment may be provided. The method may include identifying a target site of treatment within a patient's body via any known technique as would be understood by a person having ordinary skill in the art. Once the treatment site has been identified, a physician may introduce the catheter assembly into a biological conduit of the patient. The physician may maneuver the catheter assembly through the patient's body to the desired tissue location, or site at which therapeutic treatment is sought. The catheter assembly may extend from outside the body through an access pathway or site of incision. The hub, or site of bifurcation, of the catheter assembly may be positioned proximate the incision, connecting the indwelling catheter portion to external attachments.

The catheter assembly may include a steerable mechanism so that its movements can be controlled external to the patient. For example, the catheter assembly may include a steering lever disposed at the proximal end of the catheter body. The steering lever may connect to a pulley system having guide wires longitudinally extending within the catheter shaft from the distal end or tip. The physician may guide the catheter assembly to the specific anatomical region for treatment using the steerable mechanism.

Following placement of the catheter assembly, a therapeutic agent and solution may be administered through the catheter shaft to the target site. The administered therapeutic agent or solution may then be irradiated with light of a predetermined wavelength, via the catheter assembly, to cause a synergistic reaction.

Alternatively, the therapeutic solution may be dispensed into the patient separately from the catheter assembly. The catheter assembly may then be guided to the area of infusion to emit light of certain wavelengths, that when combined with the administered solution, initiates a synergistic effect. The solution may be “super-charged” by the wavelengths of light, causing the solution to eliminate or reduce microorganisms at a higher percentage than the solution acting alone.

In some exemplary embodiments, the method may further include adjusting the temperature of the therapeutic solution to improve its efficacy.

In other exemplary embodiments, the physician may utilize various external attachments to perform functions as best determined by actual operating conditions and requirements.

The catheter assembly may include securement means to stabilize the catheter assembly within the patient cavity over extended periods of time or for chronic medical procedures. Securement may be accomplished by any of a variety of known ways as would be understood by one skilled in the art. For example, the catheter may include a clip or hub anchor to removably attach the catheter hub to the skin of a patient. Securement means can also include adhesives, sutures, and/or a securement device that facilitates the affixation of an implantable catheter within a subcutaneous tunnel.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A catheter assembly comprising:
an elongated, tubular catheter body having a proximal end, a distal end, and a centrally-disposed lumen extending therebetween;
a covering attached to the distal end of the catheter body, the covering defining a generally spherical shaped tip;
at least one ancillary lumen disposed within the catheter body and extending between the proximal end and the distal end thereof;
at least one light-emitting element configured to emit light of a predetermined wavelength through the ancillary lumen; and
at least one light source that provides light of a predetermined wavelength to the light-emitting element.

2. The catheter assembly of claim 1, further comprising:
a reflective surface configured to direct light of a predetermined wavelength from the ancillary lumen to a target surface.

3. The catheter assembly of claim 1, wherein the catheter body includes a first ancillary lumen for delivering light of a predetermined wavelength to a target surface, and a second ancillary lumen for delivering therapeutic solution to the target surface.

4. The catheter assembly of claim 3, further comprising:
one or more additional ancillary lumen, each additional lumen for delivering a discrete therapeutic solution to the target surface.

5. The catheter assembly of claim 1, further comprising:
a camera disposed at the distal end of the catheter body for providing visual feedback.

6. The catheter assembly of claim 5, wherein a lens of the camera is adjustable.

7. The catheter assembly of claim 3, further comprising:
a heating element for warming therapeutic solution that passes through the second ancillary lumen.

8. The catheter assembly of claim 3, further comprising:
a cooling element for cooling therapeutic solution that passes through the second ancillary lumen.

9. The catheter assembly of claim 1, wherein the at least one light source comprises at least one of: a light emitting diode (LED) and a laser.

10. The catheter assembly of claim 1, further comprising:
an external attachment for stabilizing the catheter within a patient.

11. The catheter assembly of claim 2, wherein the at least one ancillary lumen is contiguous with an interior wall of the catheter body, and configured to direct light from the distal end of the catheter body to the reflective surface.

12. The catheter assembly of claim 1, wherein the light-emitting element comprises at least one fiber optic cable.

13. A method of providing therapeutic treatment, comprising:
introducing a catheter assembly into a biological conduit of a patient;
guiding the catheter assembly to a specific anatomical region for treatment;
administering a therapeutic solution through a lumen of the catheter assembly;
selecting a wavelength of light that reacts with the administered therapeutic solution to cause a synergistic reaction;
providing the selected wavelength of light through a lumen of the catheter assembly; and
irradiating the therapeutic solution with the light.

14. The method of claim 13, wherein the catheter assembly comprises:
an elongated, tubular catheter body having a proximal end, a distal end, and a centrally-disposed lumen extending therebetween;
a covering attached to the distal end of the catheter body, the covering defining a generally spherical shaped tip;
at least one ancillary lumen disposed within the catheter body and extending between the proximal end and the distal end thereof;
at least one light-emitting element configured to emit light of a predetermined wavelength through the ancillary lumen; and
at least one light source that provides light of a predetermined wavelength to the light-emitting element.

15. The method of claim 14, further comprising:
steering the distal tip of the catheter assembly to place the catheter assembly into an area for treatment of therapeutic solution and light.

16. The method of claim 13, further comprising:
providing at least one of a heating element and cooling element adapted to adjust the temperature of the therapeutic solution, and adjusting the temperature of the therapeutic solution.

17. The method of claim 16, wherein the heating of the therapeutic solution is performed prior to administering the therapeutic solution through the lumen of the catheter assembly.

18. The method of claim 13, further comprising:
stabilizing the catheter assembly within the biological conduit of the patient.
